(19) United States
(12) Patent Application Publication
(10) Pub. No.: US 2006/0263859 A1
(43) Pub. Date: Nov. 23, 2006

(54) Method for preparing 3'-amino-2',3'-dideoxyguanosine

(75) Inventors: Dong Ho Seong, Seoul (KR); Se Jong Han, Gunpo (KR); Seong Won Song, Anyang (KR); Byoung Jae Hahm, Gunpo (KR); Soo Young Rhie, Gunpo (KR); Jae Sung Kang, Yongin (KR)

Correspondence Address: CLARK & ELBING LLP, 101 FEDERAL STREET, BOSTON, MA, 02110, US

(73) Assignee: SAMCHULLY PHARM. CO., LTD., Seoul (KR)

(21) Appl. No.: 11/412,452

(22) Filed: Apr. 27, 2006

(30) Foreign Application Priority Data
Apr. 28, 2005 (KR) 10-2005-0035689

(51) Int. Cl. C12P 19/30; C07H 19/00

(52) U.S. Cl. 435/89; 536/27.2

(57) ABSTRACT

Disclosed therein is a method for preparing the 3′-amino-2′,3′-dideoxyguanosine, comprising the steps of: (a) treating 3′-amino-3′-deoxythymidine and 2,6-diaminopurine with a pyrimidine nucleoside phosphorylase and a purine nucleoside phosphorylase to prepare 3′-amino-2′,3′-dideoxyribosyl 2,6-diaminopurine; and (b) converting enzymatically the 3′-amino-2′,3′-dideoxyribosyl 2,6-diaminopurine with an adenosine deaminase to prepare 3′-amino-2′,3′-dideoxyguanosine. According to the present invention, 3′-amino-2′,3′-dideoxyguanosine may be obtained with very high yield in a relatively simple procedure.

### BACKGROUND OF THE INVENTION

1. Field of the Invention

The present invention relates to a novel method for preparing 3′-amino-2′,3′-dideoxyguanonine.

2. Background of the Related Art

N3′→O5′ phosphoramidates are stable in duplex and even triplex, and have higher resistance to nuclease in comparison with normal DNA or RNA. They may be used as DNA hybridization probes and primers for PCR amplification which has high selectivity to low copy RNA sequences. However, there is the big obstacle that it is very difficult to obtain amino nucleosides.

Among amino nucleosides, it is known that 3′-amino-2′,3′-dideoxyguanosine may be prepared chemically. In this method, 3′-azido-3′-deoxythymidine is converted chemically into 3′-azido-3′-deoxy-5′-O-acetylthymidine, and subsequently substituted with N²-palmitoylguanine (M. Imazawa et al., J.O.C., 43(15): 3044 (1978)). However, in this method, a production yield is low (28%), and isolation and purification of the product is difficult due to production of anomer. It is also known that 3′-amino-2′,3′-dideoxylguanosine may be produced chemoenzymatically. In this method, 3′-azido-3′-deoxythymidine is reduced chemically to 3′-amino-3′-deoxythymidine, and then 3′-amino-2′,3′-dideoxylguanosine may be produced by subsequent enzyme reaction (Galina V. Zaitseva et al., Nucleosides & Nucleotides, 13(1-3): 819 (1994)). However, in this method, a production yield (20.5%) after purification is not sufficient due to low substitution efficiency.

Therefore, there is still a need for a method for preparing 3′-amino-2′,3′-dideoxylguanosine having high production yield for industrial production.

### SUMMARY OF THE INVENTION

The inventors have studied intensively a method for preparing 3′-amino-2′,3′-dideoxyguanosine at high production yield, and as a result have completed the present invention by developing a novel enzymatic method for preparing 3′-amino-2′,3′-dideoxyguanosine using two types of enzymes.

Accordingly, it is an object of the present invention to provide a method for preparing 3′-amino-2′,3′-dideoxyguanosine.

Other objects and advantages of the present invention will become apparent from the detailed description to follow and together with the appended claims and drawings.

### BRIEF DESCRIPTION OF THE DRAWINGS

FIG. 1 illustrates an embodiment of the present invention preparing 3′-amino-2′,3′-dideoxyguanosine.

### DETALIED DESCRIPTION OF THIS INVENTION

In an aspect of this invention, there is provided a method for preparing 3′-amino-2′,3′-dideoxyguanosine of the following formula 1, comprising the steps of:

(a) treating 3′-amino-3′-deoxythymidine and 2,6-diaminopurine with a pyrimidine nucleoside phosphorylase and a purine nucleoside phosphorylase to prepare 3′-amino-2′,3′-dideoxyribosyl 2,6-diaminopurine; and

(b) enzymatically converting the 3′-amino-2′,3′-dideoxyribosyl 2,6-diaminopurine with an adenosine deaminase to prepare 3′-amino-2′,3′-dideoxyguanosine.

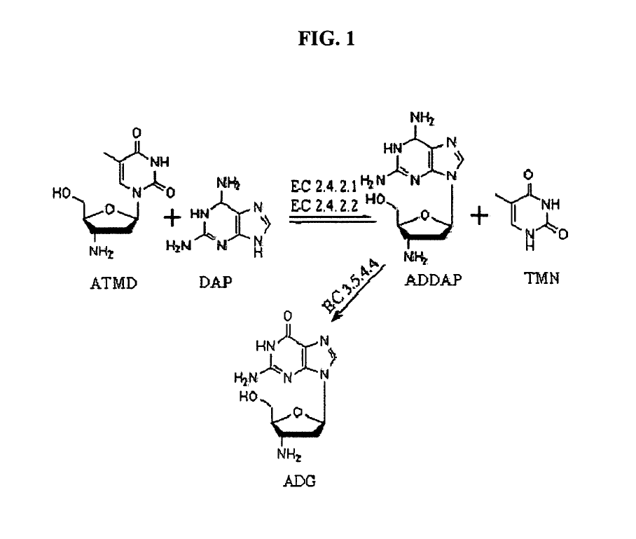

More specifically, 3′-amino-3′-deoxythymidine (Formula 2) and 2,6-diaminopurine (Formula 3) are transglycosylated with nucleoside phosphorylases to produce 3′-amino-2′,3′-dideoxyribosyl 2,6-diaminopurine (Formuls 4), and 3′-amino-2′,3′-dideoxyribosyl 2,6-diaminopurine is converted enzymatically with adenosine deaminase to prepare 3′-amino-2′,3′-dideoxyguanosine (Formula 1) (See FIG. 1).

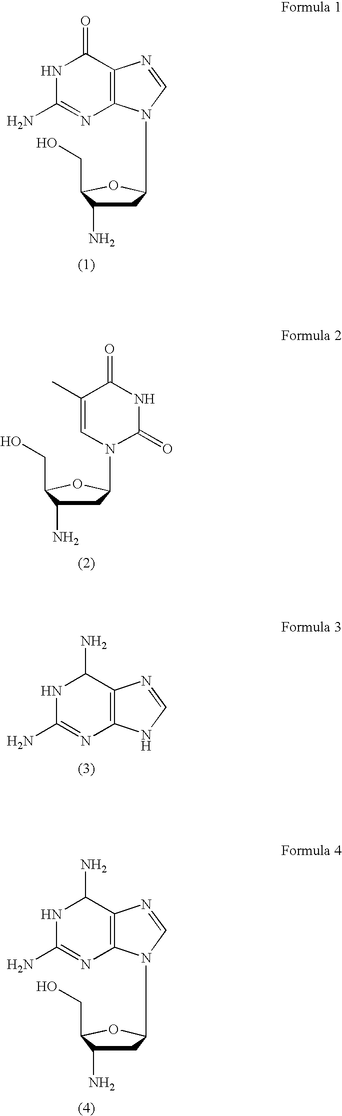

The nucleoside phosphorylases used in the present invention are a purine nucleoside phosphorylase and a pyrimidine nucleoside phosphorylase. These nucleoside phosphorylases are isolated and purified enzymes, microbial cells having the nucleoside phosphorylase activity, microbial cells genetically modified to possess the nucleoside phosphorylase activity or treatments of the microbial cells.

A nucleoside phosphorylase is a generic name of an enzyme which phosphorylases N-glycosidic bond of nucleosides in the presence of phosphoric acids. For example, in the case of a ribonucleoside, a nucleoside phosphorylase catalyzes the following reaction: Ribonucleoside+phosphoric acid (phosphate)−nucleic acid base+ribose 1-phosphate

The nucleoside phosphorylase which is largely divided into a purine nucleoside phosphorylase and a pyrimidine nucleoside phosphorylase, is ubiquitously present in various organisms, for example, mammal, aves, fish, yeast and bacteria. The reaction which these enzymes catalyze is reversible, and it has been known that various nucleoside compounds may be synthesized by using a reverse reaction.

A nucleoside phosphorylase used in the present invention means a generic name of an enzyme which catalyzes the cleavage of N-glycosidic bond of nucleosides in the presence of phosphoric acids. The present invention uses the reverse reaction of the reaction catalyzed by nucleoside phosphorylase. Enzymes which may be used in the present invention include, but not limited to, any enzyme having an activity which may synthesize 3′-amino-2′,3′-dideoxyribosyl 2,6-diaminopurine from 3′-amino-3′-deoxythymidine and 2,6-diaminopurine. There are no specific limitations for types and sources of an enzyme.

A nucleoside phosphorylase may be divided largely into a purine nucleoside phosphorylase and a pyrimidine nucleoside phosphorylase. A purine nucleoside phosphorylase includes purine nucleoside phosphorylase (EC 2.4.2.1) and guanosine phosphorylase (EC 2.4.2.15), and a pyrimidine nucleoside phosphorylase includes pyrimidine nucleoside phosphorylase (EC 2.4.2.2), uridine phosphorylase (EC 2.4.2.3), thymidine phosphorylase (EC 2.4.2.4) and deoxyuridine phosphorylase (EC 2.4.2.23).

A microorganism having a nucleoside phosphorylase activity used in the present invention means that a microorganism to have inherently a gene coding for nucleoside phosphorylase and express the enzyme. Examples of the microorganism include Nocardia, Microbacterium, Corynebacterium, Brevibacterium, Cellulomonas, Flabobacterium, Kluyvere, Micobacterium, Haemophilus, Micoplana, Protaminobacter, Candida, Saccharomyces, Bacillus, Pseudomonas, Micrococcus, Hafnia, Proteus, Vibrio, Staphyrococcus, Propionibacterium, Sartina, Planococcus, Escherichia, Kurthia, Rhodococcus, Adinetobacter, Xanthobacter, Streptomyces, Rhizobium, Salmonella, Kilebsiella, Enterobacter, Erwinia, Aeromonas, Citrobacter, Achromobacter, Agrobacterium, Arthrobacter and Pseudonocardia.

As a nucleoside phosphorylase in the present invention, genetically modified microbial cells to have the nucleoside phosphorylase activity, i.e., nucleoside phosphorylase-expressing recombinant microbial cells, may be used. These recombinant microorganisms are prepared to over-express nucleoside phosphorylase. The method for preparing recombinant microorganisms having an exogenous nucleoside phosphorylase gene, is known to in the art (Sambrook, J. et al., Molecular Cloning, A Laboratory Manual, 3rd Ed. Cold Spring Harbor Press (2001)).

Typically, vectors carrying the nucleotide sequences encoding nucleoside phosphorylase are prepared. The vector system may be constructed according to the known method in the art as described in Sambrook et al., Molecular Cloning, A Laboratory Manual, Cold Spring Harbor Laboratory Press (2001), which is incorporated herein by reference. The vector may be constructed for use in prokaryotic or eukaryotic host cells.

For example, where the vector is constructed for expression in prokaryotic cells, it generally carries a strong promoter to initiate transcription (e.g., pL^{λ} promoter, trp promoter, lac promoter, tac promoter and T7 promoter), a ribosome binding site or translation initiation and a transcription/translation termination sequence. In particular, where E. coli is used as a host cell, a promoter and an operator in operon for tryptophan biosynthesis in E. coli (Yanofsky, C., J. Bacteriol., 158:1018-1024(1984)) and a leftward promoter of phage λ (pL^{λ} promoter, Herskowitz, I. and Hagen, D., Ann. Rev. Genet., 14:399-445(1980)) may be employed as a control sequence. Where Bacillus is used as a host cell, a promoter for a gene encoding toxin protein of Bacillus thurigensis (Appl. Environ. Microbiol. 64: 3932-3938 (1998); and Mol. Gen. Genet. 250:734-741(1996)) or other promoters operable in Bacillus may be employed as a control sequence.

Numerous conventional vectors used for prokaryotic cells are known to those of skill in the art, and the selection of an appropriate vector is a matter of choice. Conventional vector used in this invention includes, but are not limited to, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, λgt4·λB, λ-Charon, λΔz1 and M13.

For example, where the expression vector is constructed for eukaryotic host cell, inter alia, animal cell, a promoter derived the genome of mammalian cells (e.g., metallothionein promoter) or mammalian virus (e.g., adenovirus late promoter; vaccinia virus 7.5K promoter, SV40 promoter, cytomegalovirus promoter and tk promoter of HSV) may be used. The vector generally contains a polyadenylation site of the transcript. The example of commercial virus-based vectors includes pcDNA 3 (Invitrogen; containing cytomegalo virus promoter and polyadenylation signal), pSI (Promega; containing SV 40 promoter and polyadenylation signal), pCI (Promega; containing containing cytomegalo virus promoter and polyadenylation signal), and pREP7 (Invitrogen; RSV promoter and SV 40 polyadenylation signal).

Where the expression vector is constructed for yeast, the promoter of the gene for phosphoglycerate kinase, glyceraldehydes-3-phosphate dehydrogenase, lactase, enolase and alcohol dehydrogenase may be used as a control sequence.

Where the expression vector is constructed for a plant cell, numerous plant-functional promoters known in the art may be used, including the cauliflower mosaic virus (CaMV) 35S promoter, the Figwort mosaic virus 35S promoter, the sugarcane bacilliform virus promoter, the commelina yellow mottle virus promoter, the light-inducible promoter from the small subunit of the ribulose-1,5-bis-phosphate carboxylase (ssRUBISCO), the rice cytosolic triosephosphate isomerase (TPI) promoter, the adenine phosphoribosyltransferase (APRT) promoter of Arabidopsis, the rice actin 1 gene promoter, and the mannopine synthase and octopine synthase promoters.

In addition, the expression vector of this invention further comprises a nucleotide sequence to conveniently purify the fusion protein expressed, which includes but not limited to, glutathione S-transferase (Pharmacia, USA), maltose binding protein (NEB, USA), FLAG (IBI, USA) and 6× His (hexahistidine; Quiagen, USA). The most preferable sequence is 6× His because it has not antigenicity and does not interfere desirable folding of the fusion protein of interest. Due to the additional sequence, the fusion protein expressed can be purified with affinity chromatography in a rapid and feasible manner.

It is preferable that the expression vector carries one or more markers which make it possible to select the transformed host, for example, genes conferring the resistance to antibiotics such as ampicillin, gentamycine, chloramphenicol, streptomycin, kanamycin, neomycin, geneticin and tetracycline, URA3 gene, genes conferring the resistance to any other toxic compound such as certain metal ions.

The vectors are transformed into a suitable host cell to prepare recombinant cells expressing nucleoside phosphorylase. The transformation can be carried out by a large number of methods known to one skilled in the art. For example, in case of prokaryotic cells as host, CaCl₂ method (Cohen, S. N. et al., Proc. Natl. Acac. Sci. USA, 9:2110-2114(1973)), Hanahan method (Cohen, S. N. et al., Proc. Natl. Acac. Sci. USA, 9:2110-2114(1973); and Hanahan, D., J. Mol. Biol., 166:557-580(1983)) and electrophoresis (Dower, W. J. et al., Nucleic. Acids Res., 16:6127-6145(1988)) can be used for transformation. Also, in case of eukaryotic cells as host, microinjection (Capecchi, M. R., Cell, 22:479(1980)), calcium phosphate precipitation (Graham, F. L. et al., Virology, 52:456(1973)), electrophoresis (Neumann, E. et al., EMBO J., 1:841(1982)), liposome-mediated transfection (Wong, T. K. et al., Gene, 10:87(1980)), DEAE-dextran treatment (Gopal, Mol. Cell Biol., 5:1188-1190(1985)), and particle bombardment (Yang et al., Proc. Natl. Acad. Sci., 87:9568-9572(1990)) can be use for transformation.

The nucleoside phosphorylase or microbial cells having an activity of the enzyme used in the present invention includes commercially available enzymes, microbial cells having an activity of the enzyme, treatments of the microbial cells, immobilizations thereof or the like. For example, the treatments of the microbial cells include acetone-dried microbial cells and lysed microbial cells prepared by mechanical disruption, sonication disruption, freeze-melting treatment, pressurization-depressurization treatment, osmometric treatment, self-digestion, cell wall digestion treatment or surfactant treatment. Further, if necessary, the treatments of the microbial cells include purification of microbial cells by ammonium sulfate or acetone precipitation and/or column chromatography.

According to a preferable embodiment of the present invention, pyrimidine necleoside phosphorylase used in the present invention is thymidine phosphorylase, more preferably E. coli-derived thymidine phosphorylase.

According to a preferable embodiment of the present invention, purine nucleoside phosphorylase used in the present invention is E. coli-derived purine nucleoside phosphorylase.

According to a preferable embodiment of the present invention, nucleoside phosphorylase used in the present invention is recombinant microbial cells as such to over-express nucleoside phosphorylase by genetic recombinant technology. Generally, it is preferable to use an isolated and purified nucleoside phosphorylase; however, considering the economic efficiency of the method for preparation, it is preferable to use nucleoside phosphorylase-over-expressing recombinant microorganisms.

According to the example of the present invention, in the event that pyrimidine nucleoside phosphorylase-over-expressing recombinant microorganisms and purine nucleoside phosphorylase-over-expressing recombinant microorganisms are cultured in medium containing a substrate (3′-amino-3′-deoxythymidine and 2,6-diaminopurine), 3′-amino-2′,3′-dideoxyribosyl 2,6-diaminopurine is produced. Mediums and culturing methods, which may be used in this procedure are known in the art (Sambrook, J. et al., Molecular Cloning, A Laboratory Manual, 3rd Ed., Cold Spring Harbor Press (2001)).

The step (a) of the present invention is a transglycosylation step. That is, thymine in 3′-amino-3′-deoxythymidine (ATMD) is substituted with 2,6-diaminopurine (DAP) in the step (a). The step (a) is preferably carried out in the presence of phosphates, for example sodium phosphate.

According to a preferable embodiment of the present invention, a step adding a base, for example sodium hydroxide to the resulting reaction product of the step (a) to inactivate the pyrimidine nucleoside phosphorylase and the purine nucleoside phosphorylase before carrying out the step (b), and dissolving the obtained 3′-amino-2′,3′-dideoxyribosyl 2,6-diaminopurine in the step (a), is further carried out.

According to a preferable embodiment of the present invention, after adding base, a step for centrifuging the resulting reaction product in the step (a) to obtain a supernatant and adding an acid, for example acetic acid to the supernatant to neutralize it, is further carried out.

After carrying out the step (a), the resulting 3′-amino-2′,3′-dideoxyribosyl 2,6-diaminopurine is converted enzymatically into 3′-amino-2′,3′-dideoxyguanosine.

Adenosine deaminase used in the present invention is isolated and purified enzyme, microbial cells having the nucleoside phosphorylase activity, microbial cells genetically transformed to possess the nucleoside phosphorylase activity or treatments of the microbial cells.

There are no specific limitations for types and sources of an adenosine deaminase. Accordingly, any adenosine deaminase which may carry out deamination of 3′-amino-2′,3′-dideoxyribosyl 2,6-diaminopurine to produce 3′-amino-2′,3′-dideoxyguanosine can be used as adenosine deaminase (EC 3.5.4.4). According to a preferable embodiment of the present invention, adenosine deaminase is Lactococcus lactis, more preferably Lactococcus lactis subsp. Lactis-derived adenosine deamimase.

According to a preferable embodiment of the present invention, the recombinant microorganism (preferably E. coli) transformed with the nucleotide sequence coding for Lactococcus lactis subsp. Lactis-derived adenosine deaminase, is used as adenosine deaminase.

Since NH₃ is generated in the step (b) due to deamination, the pH of the reaction mixture becomes increasing. Therefore, according to a preferable embodiment of the present invention, the reaction in the step (b) is carried out with maintaining the pH of the reaction liquid in the step (b) in the range of 6.8-7.8. It is possible to adjust the pH by adding an acid, for example acetic acid to the reaction liquid.

The present invention allows for the preparation of 3′-amino-2′,3′-dideoxyguanosine having much higher yield, at least 50% yield.

The following specific examples are intended to be illustrative of the invention and should not be construed as limiting the scope of the invention as defined by appended claims.

### EXAMPLES

### Example 1

### Preparation of 3′-amino-3′-deoxythymidine

4 kg of 3′-azidothymidine was stirred with 6 L of acetonitrile. To the mixture was added 4.7 kg of triphenylphosphine. The resulting mixture was stirred at room temperature for 3 hrs. And then, 4 L of distilled water was added to the reaction mixture, and the solution was stirred at room temperature for 4 hrs, and concentrated at reduced pressure. To the concentrates was added 12 L of methanol, and the solution was stirred at room temperature for 8 hrs, and filtered to produce crystals. The crystals were collected and dried to obtain 2.7 kg of 3′-amino-3′-deoxythymidine.

### Example 2

### Preparation of E. coli Uriding Phosphorylase Expression Strain

Genetic recombinant E. coli which over-express E. coli-derived uridine phosphoryolase and which is used for transglycosylation, was prepared as follows:

Eschericha coli (E. coli) JM109 strain (Promega Inc.) was seeded in 50 ml of LB medium, cultured overnight at 37° C. and centrifuged to harvest cells. Genomic DNA was isolated from the harvested E. coli with Dneasy Tissue Kit (Qiagen Inc.) and used as a PCR template.

Oligonucleotides of the following SEQ ID NO. 1 and 2 which were designed based on base sequence (Genbank Accession No. X15689, coding region base No. 163-924) of known E. coli udp gene and which were prepared by the commission manufacturer BIONICS, were used as PCR primers: 5′-CATATGTCCAAGTCTGATGTTTTTCATCTCGGC-3′ and 5′-AAGCTTTTACAGCAGACGACGCGCCGCTTCCACC-3′.

PCR reaction was carried out by using the E. cloi genome DNA as a template at 94° C. for 1 min, at 55° C. for 1 min and at 72° C. for 2 min (×30 times) in the presence of 200 μM dNTP, 20 pmol primers, 1× Taq DNA polymerase buffer and 2.5 U Taq DNA polymerase. And then, the amplified 775 bp of PCR product was visualized by agarose gel electrophoresis, and purified by using gel extraction kit (Qiagen). After DNA fragments purified by using pGEM-T easy vector system I (Promega Inc.) were ligated to pGEM-T easy vector, the clones having the desired plasmid were screened from E. coli populations obtained by transformation of JM109 E. coli cells. The screened clone having desired plasmid was referred to pGEM-EUDP.

To insert E. coli uridine phosphorylase (EUDP) gene into expression vector for E. coli, pFRPT (KR patent No. 0449639) which have been produced by the applicant of the present invention, 10 μg of pFRPT was digested in 20 μl of a reaction liquid containing 10U Nde I and 10U Hind III. The digested plasmid was assayed by agarose gel electrophoresis, and then 6.5 kb of fragment was purified by using gel extraction kit. Meanwhile, 10 μg of pGEM-EUDP was digested in 20 μl of a reaction liquid containing 10U Nde I and 10U Hind III, assayed by agarose gel electrophoresis as described above, and then 775 bp of EUDP DNA fragment was purified by using gel extraction kit. Two fragments obtain by above procedures were added to a reaction liquid containing 3U ligase and 1× ligase buffer, and reacted at 16° C. for 18 hrs. After transforming JM109 E. coli cell with the reaction liquid, the transformed JM109 E. coli cells were cultured up to E. coli populations. Plasmids were extracted from the E. coli populations, and the plasmid having the desired DNA fragment was referred to pFRPT-EUDP. The transformed E. coli strain obtained by such procedure was referred to pFRPT-EUDP/JM109.

### Example 3

### Preparation of E. coli Adenosine Deaminase Expression Strain

Genetic recombinant E. coli which over-express E. coli-derived adenosine deaminase and which is used for deamination, was prepared as follows:

Oligonucleotides of the following SEQ ID NO. 3 and 4 which were designed based on base sequence (Genbank Accession No. X59033, coding region base No. 109-1107) of known E. coli gene and which were prepared by the commission manufacturer BIONICS, were used as PCR primers: 5′-ACGGATCC-ATGATTGATACCACCCTGCCAT-3′ and 5′-GGGGTACC-TTACTTCGCGGCGACTTTTTCT-3′.

PCR reaction was carried out by using the E. cloi genome DNA prepared as described above as a template at 94° C. for 30 sec, at 55° C. for 1 min and at 72° C. for 1 min (×30 times) in the presence of 200 μM dNTP, 20 pmol primers, 1× vent DNA polymerase buffer and 2.5U vent DNA polymerase. And then, the amplified 1 kb of PCR product was visualized by agarose gel electrophoresis, and purified by using gel extraction kit (Qiagen). The purified DNA fragment was digested with Bam HI and Kpn I, and then the digested DNA fragment was ligated to plasmid expression vector pQE31 (Qiagen Inc.) digested with BamHI and KpnI. JM109 E. coli cells were transformed with the vector. The plasmid having the desired DNA fragment is referred to pQE31-ADD.

To insert E. coli adenosine deaminase (EADD) gene into expression vector for E. coli, pFRPT (KR patent No. 0449639) which have been produced by the applicant of the present invention, 10 μg of pFRPT was digested in 20 μl of a reaction liquid containing 10U Bgl II and 10U Kpn I. The digested plasmid was assayed by agarose gel electrophoresis, and then 6.45 kbp of fragment was purified by using gel extraction kit. Meanwhile, 10 μg of pQE31-EADD was digested in 20 μl of a reaction liquid containing 10U Bam HI and 10U Kpn I, assayed by agarose gel electrophoresis as described above, and then 999 bp of EADD DNA fragment was purified by using gel extraction kit. Two fragments obtain by above procedures were added to a reaction liquid containing 3U ligase and 1× ligase buffer, and reacted at 16° C. for 18 hrs. After transforming JM109 E. coli cell with the reaction liquid, the transformed JM109 E. coli cells were cultured up to E. coli populations. Plasmids were extracted from the E. coli populations, and the plasmid having the desired DNA fragment was referred to pFRPT-EADD. The transformed E. coli strain obtained by such procedure was referred to pFRPT-EADD/JM109.

### Example 4

### Preparation of Lactococcus lactis Subsp. Lactis adenosine Deaminase Expression Strain

Genetic recombinant E. coli which over-express Lactococcus-derived adenosine deaminase (LADD) and which is used for deamination, was prepared as follows:

50 ml of Lactococcus lactis subsp. Lactis strain (subdivided from KFCC; KCCM 40104) was seeded to 50 ml of TSB medium, cultured overnight at 30° C. and centrifuged to harvest cells. A genome DNA was isolated from the harvested cells by using DNeasy Tissue kit (Qiagen Inc.) and was used as PCR template.

Oligonucleotides of the following SEQ ID NO. 5 and 6 which were designed based on base sequence (Genbank Accession No. NC_{—}002662, coding region base No. 287447-288505) of known Lactococcus lactis subsp. Lactis adenosine deaminase gene and which were prepared by the commission manufacturer BIONICS, were used as PCR primers: 5′-GGATCCA-ATG AAA AGA AAA GGG AGA AAC TC-3′ and 5′-AAGCTT-CTC TGA TTA TTC AGA GAT TTT TTT G-3′.

PCR reaction was carried out by using the Lactococcus lactis subsp. Lactis genome DNA prepared as described above as a template at 94° C. for 1 min, at 50° C. for 1 min and at 72° C. for 1.5 min (×30 times) in the presence of 200 μM dNTP, 30 pmol primers, 1× Taq DNA polymerase buffer and 2.5U Taq DNA polymerase. And then, the amplified 1078 bp of PCR product was visualized by agarose gel electrophoresis, and purified by using gel extraction kit (Qiagen). The DNA fragment purified by using pGEM-T easy vector system I (Promega Inc.) was ligated to pGEM-T easy vector. After transforming JM109 E. coli cell with the vector, the transformed JM109 E. coli cells were cultured up to E. coli populations. A clone having the desired plasmid was screened from the populations, and was referred to pGEM-LADD.

For over-expression of enzyme proteins, the pGEM-LADD was digested with BamHI and Hind III, and then the digested DNA fragment was ligated to the plasmid expression vector pQE31 (Qiagen Inc.) digested with Bam HI and Hind III. JM109 E. coli cells were transformed with the vector. The plasmid having the desired DNA fragment is referred to pQE31-LADD. The transformed cells were referred to pFRPT-LADD/JM109.

### Example 5

### Preparation of E. coli Purine Nucleoside Phosphorylase Expression Strain

Genetic recombinant E. coli which over-express E. coli-derived purine nucleoside phosphoryolase and which is used for transglycosylation, was prepared as follows:

Oligonucleotides of the following SEQ ID NO. 7 and 8 which were designed based on base sequence (Genbank Accession No. M60917, coding region base No. 123-842) of known E. coli gene and which were prepared by the commission manufacturer BIONICS, were used as PCR primers: 5′-GGATCCCATGGCTACCCCACACATTAATGCA-3′ and 5′-AAGCTTTTACTCTTTATCGCCCAGCAGAAC-3′.

PCR reaction was carried out by using the E. coli genome DNA prepared as described above as a template at 94° C. for 30 sec, at 50° C. for 1 min and at 72° C. for 1 min (×30 times) in the presence of 200 μM dNTP, 20 pmol primers, 1× Taq DNA polymerase buffer and 2.5U Taq DNA polymerase. And then, the amplified 720 bp of PCR product was visualized by agarose gel electrophoresis, and purified by using gel extraction kit (Qiagen). The DNA fragment purified by using pGEM-T easy vector system I (Promega Inc.) was ligated to pGEM-T easy vector. After transforming JM109 E. coli cell with the vector, the transformed JM109 E. coli cells were cultured up to E. coli populations. A clone having the desired plasmid was screened from the populations, and was referred to pGEM-EPUNP.

To insert E. coli purine nucleoside phosphorylase (EPUNP) gene into expression vector for E. coli, pFRPT (KR patent No. 0449639) which have been produced by the applicant of the present invention, 10 μg of pFRPT was digested in 20 μl of a reaction liquid containing 10U Bgl II and 10U Hind III. The digested plasmid was assayed by agarose gel electrophoresis, and then 6.45 kbp of fragment was purified by using gel extraction kit. Meanwhile, 10 μg of pGEM-EPUNP was digested in 20 μl of a reaction liquid containing 10 U BamHI and 10 U Hind III, assayed by agarose gel electrophoresis as described above, and then 720 bp of EPUNP DNA fragment was purified by using gel extraction kit. Two fragments obtain by above procedures were added to a reaction liquid containing 3U ligase and 1× ligase buffer, and reacted at 16° C. for 18 hrs. After transforming JM109 E. coli cell with the reaction liquid, the transformed JM109 E. coli cells were cultured up to E. coli populations. Plasmids were extracted from the E. coli populations, and the plasmid having the desired DNA fragment was referred to pFRPT-EPUNP. The transformed E. coli strain obtained by such procedure was referred to pFRPT-EPUNP/JM109.

### Example 6

### Preparation E. coli Thymidine Phosphorylase Expression Strain

Genetic recombinant E. coli which over-expresses E. coli-derived thymidine phosphorylase and which is used for transglycosylation, was prepared as follows:

Oligonucleotides of the following SEQ ID NO. 9 and 10 which were designed based on base sequence (Genbank Accession No. U14003, coding region base No. 1-1323) of known E. coli gene and which were prepared by the commission manufacturer BIONICS, were used as PCR primers: 5′-CCATGGTTGTTTCTCGCACAAGAACT-3′ and 5′-GATATCTTATTCGCTGATACGGCGATAG-3′.

PCR reaction was carried out by using the E. coli genome DNA prepared as described above as a template at 94° C. for 1 min, at 55° C. for 1 min and at 72° C. for 2 min (×30 times) in the presence of 200 μM dNTP, 20 pmol primers, 1× Taq DNA polymerase buffer and 2.5U Taq DNA polymerase. And then, the amplified 1.3 kbp of PCR product was visualized by agarose gel electrophoresis, and purified by using gel extraction kit (Qiagen). The DNA fragment purified by using pGEM-T easy vector system I (Promega Inc.) was ligated to pGEM-T easy vector. After transforming JM109 E. coli cell with the vector, the transformed JM109 E. coli cells were cultured up to E. coli populations. A clone having the desired plasmid was screened from the populations, and was referred to pGEM-TMDP.

To insert E. coli thymidine phosphorylase (TMDP) gene into expression vector for E. coli pFRPT (KR patent No. 0449639) which have been produced by the applicant of the present invention, 10 μg of pFRPT was digested in 20 μl of a reaction liquid containing 10U NcoI and 10U EcoRV. The digested plasmid was assayed by agarose gel electrophoresis, and then 6.45 kbp of fragment was purified by using gel extraction kit. Meanwhile, 10 μg of pGEM-TMDP was digested partially in 20 μl of a reaction liquid containing 10U NcoI and 10U EcoRV, assayed by agarose gel electrophoresis as described above, and then 1.3 kbp of TMDP DNA fragment was purified by using gel extraction kit. Two fragments obtain by the above procedures were added to a reaction liquid containing 3U ligase and 1× ligase buffer, and reacted at 16° C. for 18 hrs. After transforming JM109 E. coli cell with the reaction liquid, the transformed JM109 E. coli cells were cultured up to E. coli populations. Plasmids were extracted from the E. coli populations, and the plasmid having the desired DNA fragment was referred to pFRPT-TMDP. The transformed E. coli strain obtained by such procedure was referred to PFRPT-TMDP/JM109.

### Example 7

### Preparation of E. coli pFRPT-EUDP/JM109 Wet Microbial Cells

To 25 ml of a sterilized medium (contained in 250 ml Erlenmeyer flask) containing 30 ug/ml kanamycin-containing 0.5% yeast extracts (Difco), 0.7% beef extracts (Difco), 1.0% peptone (Difco) and 0.3% sodium chloride was seeded 1 Pt loop of E. coli pFRPT-EUDP/JM109. The culture was carried out overnight at 37° C. with shaking at 240 rpm. And then, 2 ml of the culture broth was seeded sterily to 200 ml of the same medium as the medium contained in Erlenmeyer flask. The culture was carried out at 37° C. with shaking at 240 rpm. When absorption was 0.8, IPTG was added to 1 mM of a concentration. Further, shaking culture was carried out for 3 hrs. The resulting culture broth was centrifuged at 8000 rpm for 10 min, and washed with 20 ml of 10 mM phosphate buffer. The resulting product was used as enzyme source of uridine phosphorylase derived from E. coli.

### Example 8

### Preparation of E. coli pFRPT-EADD/JM109 Wet Microbial Cells

To 25 ml of a sterilized medium (contained in 250 ml Erlenmeyer flask) containing 30 μg/ml kanamycin-containing 0.5% yeast extracts (Difco), 0.7% beef extracts (Difco), 1.0% peptone (Difco) and 0.3% sodium chloride was seeded 1 Pt loop of E. coli pFRPT-EADD/JM109. The culture was carried out overnight at 37° C. with shaking at 240 rpm. And then, 2 ml of the culture broth was seeded sterily to 200 ml of the same medium as the medium contained in Erlenmeyer flask. The culture was carried out at 37° C. with shaking at 240 rpm. When absorption was 0.8, IPTG was added to 1 mM of a concentration. Further, shaking culture was carried out for 3 hrs. The resulting culture broth was centrifuged at 8000 rpm for 10 min, and washed with 20 ml of 10 mM phosphate buffer. The resulting product was used as enzyme source of adenosine phosphorylase derived from E. coli.

### Example 9

### Preparation of E. coli pFRPT-LADD/JM109 Wet Microbial Cells

To 25 ml of a sterilized medium (contained in 250 ml Erlenmeyer flask) containing 30 μg/ml kanamycin-containing 0.5% yeast extracts (Difco), 0.7% beef extracts (Difco), 1.0% peptone (Difco) and 0.3% sodium chloride was seeded 1 Pt loop of E. coli pFRPT-LADD/JM109. The culture was carried out overnight at 37° C. with shaking at 240 rpm. And then, 2 ml of the culture broth was seeded sterily to 200 ml of the same medium as the medium contained in Erlenmeyer flask. The culture was carried out at 37° C. with shaking at 240 rpm. When absorption was 0.8, IPTG was added to 1 mM of a concentration. Further, shaking culture was carried out for 3 hrs. The resulting culture broth was centrifuged at 8000 rpm for 10 min, and washed with 20 ml of 10 mM phosphate buffer. The resulting product was used as enzyme source of adenosine deaminase derived from Lactococcus.

### Example 10

### Preparation of E. coli pFRPT-EPUNP/JM109 Wet Microbial Cells

To 25 ml of sterilized medium (contained in 250 ml Erlenmeyer flask) containing 30 μg/ml kanamycin-containing 0.5% yeast extracts (Difco), 0.7% beef extracts (Difco), 1.0% peptone (Difco) and 0.3% sodium chloride was seeded 1 Pt loop of E. coli pFRPT-EPUNP/JM109. The culture was carried out overnight at 37° C. with shaking at 240 rpm. And then, 2 ml of the culture broth was seeded sterily to 200 ml of the same medium as the medium contained in Erlenmeyer flask. The culture was carried out at 37° C. with shaking at 240 rpm. When absorption was 0.8, IPTG was added to 1 mM of a concentration. Further, shaking culture was carried out for 3 hrs. The resulting culture broth was centrifuged at 8000 rpm for 10 min, and washed with 20 ml of 10 mM phosphate buffer. The resulting product was used as enzyme source of thymidine phosphorylase derived from E. coli.

### Example 11

### Preparation of E. coli pFRPT-ETDP/JM109 Wet Microbial Cells

To 25 ml of a sterilized medium (contained in 250 ml Erlenmeyer flask) containing 30 μg/ml kanamycin-containing 0.5% yeast extracts (Difco), 0.7% beef extracts (Difco), 1.0% peptone (Difco) and 0.3% sodium chloride was seeded 1 Pt loop of E. coli pFRPT-ETDP/JM109. The culture was carried out overnight at 37° C. with shaking at 240 rpm. And then, 2 ml of the culture broth was seeded sterily to 200 ml of the same medium as the medium contained in Erlenmeyer flask. The culture was carried out at 37° C. with shaking at 240 rpm. When absorption was 0.8, IPTG was added to 1 mM of a concentration. Further, shaking culture was carried out for 3 hrs. The resulting culture broth was centrifuged at 8000 rpm for 10 min, and washed with 20 ml of 10 mM phosphate buffer. The resulting product was used as enzyme source of thymidine phosphorylase derived from E. coli.

### Example 12

### Preparation of 3′-amino-2′,3′-dideoxyribosyl 2,6-diaminopurine

To a substrate solution containing 0.15 g of 2,6-diaminopurine (DAP), 0.22 g of 3′-amino-3′-deoxythymidine (ATMD), 3 ml of distilled water and 0.5 ml of 1 N sodium phosphate buffer (pH 7.5) were added 0.2 g of E. coli pFRPT-EPUNP/JM109 wet microbial cells and 0.2 g of E. coli pFRPT-ETDP/JM109 wet microbial cells. The reaction was carried out at 50° C. for 2 days with agitation. According to the analysis results obtained from HPLC analysis, the yield of 3′-amino-2′,3′-dideoxyribosyl 2,6-diaminopurine was 50.45%.

The analysis after the reaction was carried out by using HPLC under the conditions: column; Inersil ODS-3 (5 micrometer, diameter 4.6 mm, length 150 mm, GL science), moving phase; 4% methanol-containing 10 mM sodium phosphate buffer (pH 8.0), detection; UV 254 nm absorption.

### Example 13

### Preparation of 3′-amino-2′,3′-dideoxyribosyl 2,6-diaminopurine

To a substrate solution containing 0.15 g of 2,6-diaminopurine (DAP), 0.22 g of 3′-amino-3′-deoxythymidine (ATMD), 3 ml of distilled water and 0.5 ml of 1 N sodium phosphate buffer (pH 7.5) were added 0.2 g of E. coli pFRPT-EPUNP/JM109 wet microbial cells and 0.2 g of E. coli pFRPT-ETDP/JM109 wet microbial cells. The reaction was carried out at 50° C. for 2 days with agitation. According to the analysis results obtained from HPLC analysis, the yield of 3′-amino-2′,3′-dideoxyribosyl 2,6-diaminopurine was 77.18%.

### Example 14

### Preparation of 3′-amino-2′,3′-dideoxyribosyl 2,6-diaminopurine

To a substrate solution containing 7.5 g of 2,6-diaminopurine (DAP), 18.1 g of 3′-amino-3′-deoxythymidine (ATMD), 30 ml of distilled water and 3 g of disodium hydrogen phosphate were added 5 g of E. coli pFRPT-EPUNP/JM109 wet microbial cells and 5 g of E. coli pFRPT-ETDP/JM109 wet microbial cells. The reaction was carried out at 50° C. for 40 hrs with agitation. According to the analysis results obtained from HPLC analysis, the yield of 3′-amino-2′,3′-dideoxyribosyl 2,6-diaminopurine was 96.28%.

### Example 15

### Preparation of 3′-amino-2′,3′-dideoxyguanosine

To the reaction product of example 14 was added 3.5 g of sodium hydroxide to solublize the product. After centrifuging the solution, the obtained supernatant was divided into two portions. After the supernatants were neutralized with acetic acid, 3 g of E. coli pFRPT-EADD/JM109 wet microbial cells was added to the neutralized supernatants. The reaction was carried out at 40° C. for 29 hrs with maintaining pH 7.5 to prepare 3′-amino-2′,3′-dideoxyguanosine. In this case, a reaction rate was 88.5%.

The reaction rate was calculated with the following equation:
Reaction rate=(moles of 3′-amino-2′,3′-dideoxyguanosine on completion of the reaction÷moles of 3′-amino-2′,3′-dideoxyribosyl 2,6-diaminopurine on onset of the reaction)×100

### Example 16

### Preparation of 3′-amino-2′,3′-dideoxyguanosine

To the reaction product of example 14 was added 3.5 g of sodium hydroxide to solublize the product. After centrifuging the solution, the obtained supernatant was divided into two portions. After the supernatants were neutralized with acetic acid, 3 g of E. coli pFRPT-LADD/JM109 wet microbial cells was added to the neutralized supernatants. The reaction was carried out at 40° C. for 29 hrs with maintaining pH 7.5 to prepare 3′-amino-2′,3′-dideoxyguanosine. In this case, the reaction rate was 98.97%.

### Example 17

### Screening of Suitable Base Donor

To a substrate solution (pH 7.5) containing 1 mM 3′-amino-3′-deoxythymidine (ATMD), 1 mM base donor and 5 ml of 0.1 M sodium phosphate buffer were added 0.3 g of E. coli pFRPT-EPUNP/JM109 wet microbial cells, 0.3 g of E. coli pFRPT-ETDP/JM109 wet microbial cells. The reaction was carried out at 50° C. for 42 hrs with agitation.

The nucleoside yield was calculated according to the following equation:
Nucleoside yield (%)=(moles of produced nucleoside/moles of initial base donor)×100

The results were shown in the following Table 1.

TABLE 1

| Base donor | Product | Yield(%) |
|---|---|---|
| Guanine | 3′-amino-2′,3′-dideoxyguanosine | 0.2% |
| Guanosine | 3′-amino-2′,3′-dideoxyguanosine | 38.3% |
| 2,6- | 3′-amino-2′,3′-dideoxyribosyl 2,6- | 73.7% |
| diaminopurine | diaminopurine | |

### Example 18

### Mass Production of 3′-amino-2′,3′-dideoxyguanosine

After 3 L of a substrate solution (pH 7.5) containing 252 g of 2,6-diaminopurine (DAP), 362 g of 3′-amino-3′-deoxythymidine (ATMD) and 90 g of disodium hydrogen phosphate was admixtured with 150 g of E. coli pFRPT-EPUNP/JM109 wet microbial cells and 150 g of E. coli pFRPT-ETDP/JM109 wet microbial cells, the resulting mixture was reacted in 5 L fermenter (BIOFLO 3000, NBS) at 50° C. for 36 hrs with agitation at 100 rpm. In this case, a yield of 3′-amino-2′,3′-dideoxyribosyl 2,6-diaminopurine (ADDAP) was 79%.

To the reaction liquid was added 90 g of sodium hydroxide for solublization. After centrifugation, the supernatant was collected. The precipitates were washed with 1 L of distilled water. To the mixed solution of the supernatant and the washing solution was added 85 g of acetic acid, and then added 100 g of E. coli pFRPT-LADD/JM109 wet microbial cells. The reaction was carried out at 40° C. for 5 hrs with maintaining pH 7 by using acetic acid. The deamination yield of 3′-amino-2′,3′-dideoxyribosyl 2,6-diaminopurine (ADDAP) was 95.1%. The yield of reaction for 3′-amino-2′,3′-dideoxyguanosine (before purification) was about 75.1%.

To the reaction liquid was added 350 ml of 10 N sodium hydroxide. After agitating the reaction liquid at room temperature for 3 hrs, the reaction liquid was centrifuged to collect the supernatant. The precipitate was washed with 0.5 L of purified water. The mixed solution of the supernatant and the washing solution was neutralized with HCl, and cooled with ice. After agitation for 3 hrs, the solution was centrifuged to collect crude 3′-amino-2′,3′-dideoxyguanosine. After adding 0.5 L of distilled water to the crude 3′-amino-2′,3′-dideoxyguanosine, the resulting solution was centrifuged to collect the precipitates. After drying, 268 g of 3′-amino-2′,3′-dideoxyguanosine (ADG) was obtained. The purity of 3′-amino-2′,3′-dideoxyguanosine (ADG) was 90.92% and the yield (after purification) was 57.11%.

¹H NMR(DMSO-d₆): 7.88(s, 1H), 6.50(br s, 2H), 6.05(t, 1H), 4.89(br s, 1H), 3.50(m, 6H), 2.40(m, 1H), 2.10(m, 1H)

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A method for preparing 3′-amino-2′,3′-dideoxyguanosine represented by the following formula 1, comprising the steps of:
(a) treating 3′-amino-3′-deoxythymidine and 2,6-diaminopurine with pyrimidine nucleoside phosphorylase and purine nucleoside phosphorylase to prepare 3′-amino-2′,3′-dideoxyribosyl 2,6-diaminopurine; and
(b) converting enzymatically the 3′-amino-2′,3′-dideoxyribosyl 2,6-diaminopurine with adenosine deaminase to prepare 3′-amino-2′,3′-dideoxyguanosine.

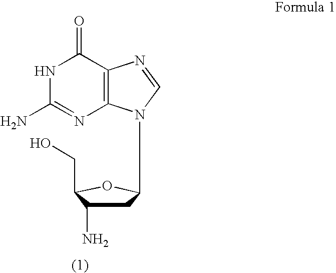

2. The method according to claim 1, wherein the pyrimidine nucleoside phosphorylase and purine nucleoside phosphorylase are selected from the group consisting of isolated and purified enzymes, microbial cells having nucleoside phosphorylase activity, microbial cells having nucleoside phosphorylase activity by a genetic recombination, and treatments of the microbial cells.

3. The method according to claim 1, wherein the adenosine deaminase is selected from the group consisting of isolated and purified enzymes, microbial cells having deaminase activity, microbial cells having deaminase activity by a genetic recombination, and treatments of the microbial cells.

4. The method according to claim 1, wherein the pyrimidine nucleoside phosphorylase is thymidine phosphorylase.

5. The method according to claim 4, wherein the thymidine phosphorylase is E. coli thymidine phosphorylase.

6. The method according to claim 1, wherein the purine nucleoside phosphorylase is E. coli purine nucleoside phosphorylase.

7. The method according to claim 1, wherein the adenosine deaminase is the adenosine deaminase of Lactococcus lactis.

8. The method according to claim 1, wherein the step (a) is carried out in the presence of a phosphate.

9. The method according to claim 1, further comprising adding a base to the resulting reaction product of the step (a) to inactivate the pyrimidine nucleoside phosphorylase and the purine nucleoside phosphorylase before carrying out the step (b), and solubilizing the 3′-amino-2′,3′-dideoxyribosyl 2,6-diaminopurine obtained in the step (a).

10. The method according to claim 9, further comprising centrifuging the resulting reaction product of the step (a) after adding the base to obtain a supernatant and adding an acid to the supernatant to neutralize the supernatant.

11. The method according to claim 1, wherein the step (b) is carried out with maintaining the pH of the reaction liquid in the range of 6.8-7.8.


SEQUENCE LISTING (10 sequences)

SEQ ID NO: 1 - DNA, 33 bp, Artificial Sequence
  primer for uridine phosphorylase
catatgtccaagtctgatgtttttcatctcggc

SEQ ID NO: 2 - DNA, 34 bp, Artificial Sequence
  primer for uridine phosphorylase
aagcttttacagcagacgacgcgccgcttccacc

SEQ ID NO: 3 - DNA, 30 bp, Artificial Sequence
  primer for adenosine deaminase
acggatccatgattgataccaccctgccat

SEQ ID NO: 4 - DNA, 30 bp, Artificial Sequence
  primer for adenosine deaminase
ggggtaccttacttcgcggcgactttttct

SEQ ID NO: 5 - DNA, 30 bp, Artificial Sequence
  primer for adenosine deaminase
ggatccaatgaaaagaaaagggagaaactc

SEQ ID NO: 6 - DNA, 31 bp, Artificial Sequence
  primer for adenosine deaminase
aagcttctctgattattcagagatttttttg

SEQ ID NO: 7 - DNA, 31 bp, Artificial Sequence
  primer for purine nucleoside phosphorylase
ggatcccatggctaccccacacattaatgca

SEQ ID NO: 8 - DNA, 30 bp, Artificial Sequence
  primer for purine nucleoside phosphorylase
aagcttttactctttatcgcccagcagaac

SEQ ID NO: 9 - DNA, 26 bp, Artificial Sequence
  primer for thymidine phosphorylase
ccatggttgtttctcgcacaagaact

SEQ ID NO: 10 - DNA, 28 bp, Artificial Sequence
  primer for thymidine phosphorylase
gatatcttattcgctgatacggcgatag